(19) United States
(12) Patent Application Publication
(10) Pub. No.: US 2015/0309184 A1
(43) Pub. Date: Oct. 29, 2015

(54) COMMUNICATING RADIOISOTOPE DOSAGE

(71) Applicant: CAPINTEC, INC., Ramsey, NJ (US)

(72) Inventors: John VISCOVIC, Park Ridge, NJ (US); Mary Anne YUSKO, Pittsburgh, PA (US)

(21) Appl. No.: 14/698,814

(22) Filed: Apr. 28, 2015

Related U.S. Application Data
(60) Provisional application No. 61/985,057, filed on Apr. 28, 2014.

(51) Int. Cl. G01T 1/02; G01T 7/02; A61N 5/10; G01T 1/185

(52) U.S. Cl. CPC G01T 1/02; G01T 1/185; G01T 7/02; A61N 5/10

(57) ABSTRACT

A method for measuring nuclear radiation in a material includes converting radiation from a material that has been inserted into a chamber into an electrical signal corresponding to an extent of nuclear radiation in the material. An electronic circuit provides to the chamber, and receives an electrical signal from the chamber in response to the presence of radiation in the material. A processor receives the electronic signal from the chamber, and uses software to process the digital data, store it, and communicate it over an electronic network. The processor hosts a web server interface which is operative to generate and host pages for access through the network, where the pages corresponding to the processed digital data.

### CROSS REFERENCE TO RELATED APPLICATIONS

This application claims the benefit of related U.S. Provisional Patent Application No. 61/985,057, filed Apr. 28, 2014, the contents of which are incorporated herein by reference in their entirety.

### FIELD OF THE DISCLOSURE

The disclosure relates to a system and method for measuring radioactivity in a sample, and more particularly to communicating therapeutic dose calibration using a network.

### BACKGROUND OF THE DISCLOSURE

Dose calibrators are used to measure radiation in a sample. An output display presents information pertaining to the radiation measured, and a key panel can be provided for enabling input of measurement parameters. Dose Calibrators can be used for dose measurements in therapeutic applications, or can be used in a ‘hotlab’ for a variety of purposes.

One form of calibrator uses an ionization chamber which measures charges created by interactions between incident radiation and a fill gas within the chamber. A voltage potential is applied between the electrodes to create an electric field in the gas. When the gas is ionized by radiation, ion-pairs are created, wherein positive ions and dissociated electrons move to the electrode of the opposite polarity under the influence of the electric field. The charge thus created is measured and correlated to the radiation emitted.

### SUMMARY OF THE DISCLOSURE

In an embodiment of the disclosure, a method for measuring nuclear radiation in a material, comprises converting radiation from the material that has been inserted into a chamber into an electrical signal corresponding to an extent of nuclear radiation in the material; using a first electronic circuit connected to the chamber for powering the chamber with an electrical signal, and for receiving an electrical signal from the chamber in response to the presence of radiation in the material; processing the received electronic signal using an electronic processor executing software stored on non-transitory media, the software operative to process the digital data; storing the processed digital data using a data storage device connected to the processor; connecting the processor to a electronic communication network; and hosting a web server, using the processor and software, the web server operative to generate and host pages for access through the network, the pages corresponding to the processed digital data.

In various embodiments thereof, the processor is connected to an electronic circuit operative to communicate with an Ethernet network; the network is an Ethernet network; the network is a TCP/IP network; the network is a USB Network the method further includes providing radiation shielding positioned between the chamber on a first side, and the first electronic circuit and processor on a second side opposite the first side; the network is an Ethernet network, and further includes a circuit for obtaining electrical energy sufficient for the operation of the chamber, the first electronic circuit, and the processor, when the Ethernet circuit is connected to a circuit providing Power Over Ethernet (PoE); and/or the material is a therapeutic dosage.

In another embodiment of the disclosure, a system for measuring nuclear radiation in a material comprises a chamber into which the material may be inserted, the chamber configured to convert an extent of nuclear radiation in the material into a corresponding electrical signal; a first electronic circuit connected to the chamber and including a circuit for powering the chamber with an electrical signal, and a circuit for receiving an electrical signal from the chamber in response to the presence of radiation in the material and for generating an output signal; a second electronic circuit connected to the first electronic circuit and including: an electronic processor operative to process the output signal into processed data; data storage connected to the processor and operative to store processed digital data; an electronic communication network connected to the electronic processor; and software stored on non-transitory media, the software executable by the processor and configured for: software executable upon the electronic processor and configured to provide a web server operative to generate and host pages for access through the network, the pages corresponding to the processed digital data.

In various embodiments thereof, the software is further configured for generating one or more pages with the web server enabling a configuration of the system to allow or deny access to hosted pages of the system based upon a network address of one or more computers attempting to access the hosted pages using the network; the circuit for receiving the electrical signal from the chamber further includes circuitry for converting the received electrical signal to digital data, the digital data corresponding to the output signal; the network is a Power Over Ethernet (PoE) network, and where the first and second circuit derive all required operating power through power provided by the PoE network; the system further includes radiation shielding positioned between the chamber on a first side, and the first and second electronic circuits on a second side opposite to the first side; the system further includes a circuit for obtaining electrical energy sufficient for the operation of the system when the network is a circuit providing Power Over Ethernet (PoE); the system further includes a USB circuit operative to enable the processor to communicate by USB; and/or the system further includes a USB circuit operative to obtain electrical energy sufficient for the operation of the system when the USB circuit is connected to a source of power.

In other embodiments thereof, the first electronic circuit includes iometer circuitry; the circuit for receiving the electrical signal includes input amplifier circuitry; the circuit for powering the chamber includes high voltage power supply circuitry; and/or the first circuit and the second circuit are connected by a serial interface.

### BRIEF DESCRIPTION OF THE DRAWINGS

A more complete understanding of the present disclosure, and the attendant advantages and features thereof, will be more readily understood by reference to the following detailed description when considered in conjunction with the accompanying drawings wherein:

FIG. 1 is a system of the disclosure including a chamber and electronics positioned within the chamber, connected to a prior art PoE adapter and computer;

FIG. 2 is an exploded perspective view of the system of FIG. 1;

FIG. 3 is a diagrammatic view of the processor board illustrated in FIG. 2;

FIG. 4 is a diagrammatic view of the iometer board illustrated in FIG. 2;

FIG. 5 is a diagram of the hardware architecture of the system of FIG. 1;

FIG. 6 is a diagram of the software architecture of the system of FIG. 1;

FIG. 7 is a screen capture of a main screen generated by the system of FIG. 1;

FIG. 8 is a screen capture of an IP Setup screen generated by the system of FIG. 1;

FIG. 9 is a screen capture of an IP Access screen generated by the system of FIG. 1;

FIG. 10 is a screen capture of a Setup User Nuclide screen generated by the system of FIG. 1; and

FIG. 11 is a computing system useable with a system of the disclosure, or illustrating components which can be included in system 100.

### DETAILED DESCRIPTION OF THE DISCLOSURE

As required, detailed embodiments are disclosed herein; however, it is to be understood that the disclosed embodiments are merely examples and that the systems and methods described below can be embodied in various forms. Therefore, specific structural and functional details disclosed herein are not to be interpreted as limiting, but merely as a basis for the claims and as a representative basis for teaching one skilled in the art to variously employ the present subject matter in virtually any appropriately detailed structure and function. Further, the terms and phrases used herein are not intended to be limiting, but rather, to provide an understandable description of the concepts.

The terms “a” or “an”, as used herein, are defined as one or more than one. The term plurality, as used herein, is defined as two or more than two. The term another, as used herein, is defined as at least a second or more. The terms “including” and “having,” as used herein, are defined as comprising (i.e., open language). The term “coupled,” as used herein, is defined as “connected,” although not necessarily directly, and not necessarily mechanically.

In accordance with the disclosure, a dosimetry system 100 includes an ion chamber 200 and associated electronics 300, collectively configured to provide sufficient speed and accuracy as required to measure and prepare doses for therapeutic benefit in a reliable and timely manner. System 100 can further measure radiation in a material for any other purpose, including for example diagnostic purposes in medice or other fields, and for any purpose which may be required in a laboratory or industrial setting. System 100 provides a web driven interface that is easy to learn and use, as described herein. Chamber 200 can include, for example, a CAPINTEC high pressure well design, a PET, or a 2 atmosphere chamber, or any other device which is operative to electrically indicate a charge state and current flow in response to the presence of nuclear radiation. In an embodiment, chamber 200 is capable of measuring a dose as high as 250 GBq (6 Ci) with a resolution of 0.001 MBq (0.01 μCi). In an example embodiment, chamber 200 is a thin wall, deep well, high pressure type, having a fill gas of 12 atm ultra pure argon, 5 atmosphere PET.

An Ethernet interface 310 for receiving data and power forms a part of electronics 300, and is configured to connect to a LAN, WAN, or other network, for example an IP based network, such as the Internet. An Ethernet connector 312, for example an RJ45 connector, is provided, although other connector configurations can be used. Any of a wide variety of network topologies currently known or hereinafter developed can be supported, including, for example, 10BASE-T, 100BASE-T, 1000BASE-T, or 10GBASE-T. The Ethernet interface can support an 8P8C or RJ45 connection, and can use any cabling from Cat3 to Cat7, inclusive, or any other suitable connector or cabling known or hereinafter developed.

System 100 can further additionally or alternatively include a USB interface 320 for receiving data and power, as part of electronics 300, and is configured to connect by a USB connector 322 to a USB hub or network, and to a USB enabled device, for example a personal computer. USB standards 1 to 3 can be supported, and A-Type, B-Type, or C-Type in standard, mini or micro sizes, or any connector types hereinafter developed. Alternatively, system 100 can support an APPLE LIGHTNING OR THUNDERBOLT connection which can perform at least the same functions as described for USB herein. The USB or Lightning connection can alternatively or additionally provide power to electronics 300 and chamber 200 as described herein for PoE.

System 100 can additionally or alternatively provide for connection to a printer 408 either by a direct wired connection using any known method, or via any known wireless method. Examples include connection to a printer using Ethernet interface 310, or USB circuit 320. Through the USB and/or Ethernet interface 310, 320, system 100 can receive and install software upgrades, and can connect to alternative ion chambers in a plug and play manner. In addition, system 100 can be controlled through interface 310 or 320, and can present data remotely.

In an embodiment, electronics 300 includes a processor 302, associated data storage 304, and software 330 (not illustrated) for hosting a browser interface transmitted through interface 310 and/or 320 through a LAN or WAN interface, to a browser application executing upon any computer device, including, for example, a computing device 400, which can include any of a desktop computer, or a tablet, laptop, smartphone, or wearable computing device. The hosted interface can include html, javascript, java, or any other browser executable software currently known or hereinafter developed, which is transmitted between electronics 300 and a browser application, such as INTERNET EXPLORER, FIREFOX, CHROME, or any other browser executable on another computing device. In this respect, electronics 300 can include the functionality of an internet web server, and as such, can store data in a database physically present within storage 304, or within a database stored elsewhere on the LAN or WAN to which system 100 is connected, for example within a local server, or within the ‘cloud’. The data stored includes data derived from measurements conducted within chamber 200, as well as data provided by a user of the browser interface, and in some embodiments, data derived from other sources, for example data from a certification or testing authority.

By enabling a browser interface, system 100 can enable a display that can be set by a user of system 100. More particularly, the size and resolution of a browser window display 340 is controlled by a local browser application and/or local hardware associated with the computing device 400, and consequently, can be set to a size easily viewable at a distance.

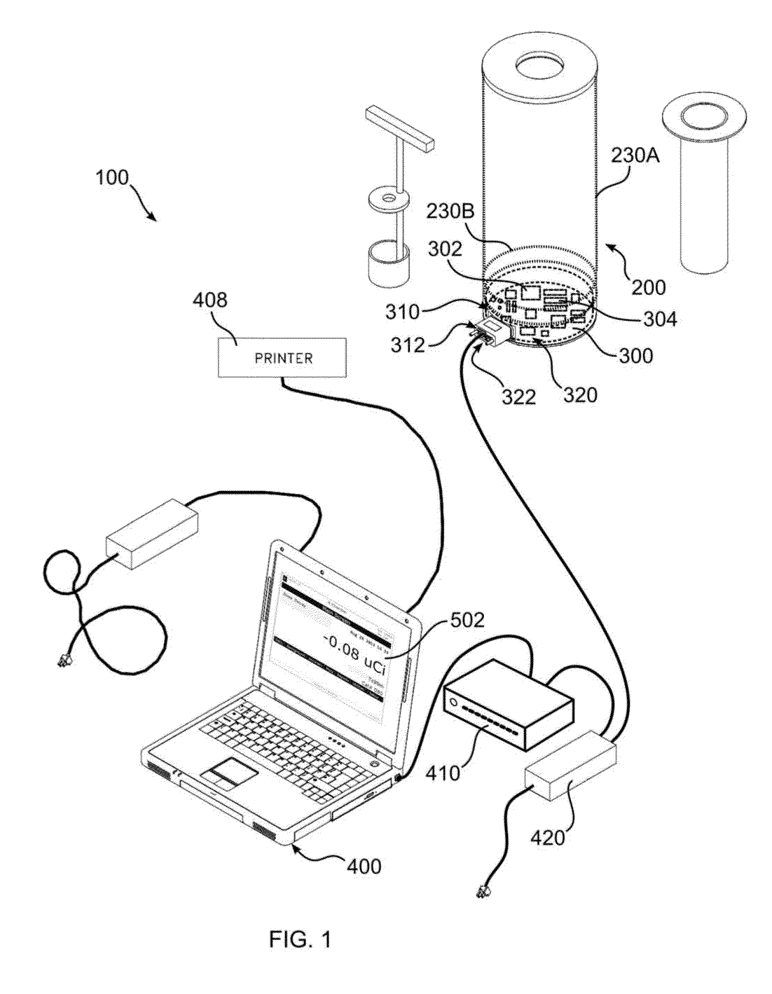

With further reference to FIG. 1, a Power over Ethernet (POE) adapter 420 transmits sufficient power through Ethernet interface 310 to power chamber 200, including powering the ionization and detection process, as well as the advanced communication functionality of the disclosure detailed herein. In FIG. 1, a midspan or PoE injector type adapter is shown, wherein a first connection is made from adapter 420 to electronics 300 within chamber 200, and a second connection is made from adapter 420 to a remainder of the network. In the embodiment of FIG. 1, PoE adapter 420 is connected to a router or switch 410, although PoE adapter 420 could alternatively be connected directly to computing device 400. The introduction of a power signal onto the same wires that carry the data signal, and/or extra wires within the cable, does not interfere with the transmitted data signal, if carried out in accordance with engineering standards, including, for example, the IEEE 802.3 standard, although other standards may be followed. In an embodiment, PoE adapter uses 100-240 VAC (50/60 Hz) and 3 A max during operation of chamber 200 and electronics 300. Chamber 200, including electronics 300, can be powered by PoE from USB using 5Vdc and 0.5 A, and from PoE from any source using 48Vdc and 0.35 A.

Switch 410 can include a wireless transmitter, for example for WiFi transmission, thereby enabling compatible wireless devices configured to support IP based browsing to communicate with system 100. Alternatively, switch 410 can be connected to a wireless transmitter (not shown), or computing device 400 can share a wired connection to thereby increase the scope of devices which can communicate with system 100. In an alternative embodiment, PoE adapter can be eliminated, and switch 410 can be a PoE type network switch or router, and can inject power into, at least, the port to which electronics 300 are connected.

Regardless of where power is injected into the Ethernet cabling, chamber 200 does not require a power connection separate from that provided by PoE. In accordance with the disclosure, PoE is applied to power the communication and processing functions of system 100, and also to power the operation of chamber 200, including the anode and cathode, and detection and measurement electronics associated with chamber 200. It should be understood that electronics associated with chamber 200 in the prior art may be partially or entirely subsumed within electronics 300 of the disclosure.

Chamber 200 wall can be fabricated, for example, with aluminum or stainless steel, and may be internally coated with an EMI (Electromagnetic Interference) blocking, and/or an EMC (Electromagnetic Compatibility) material. Lead shielding 230A, 230B, or other material that is highly effective at blocking radiation, surrounds chamber 200, and protects circuit boards 350, 352 and other components of electronics 300 from a negative impact of radiation upon the performance or reliability thereof.

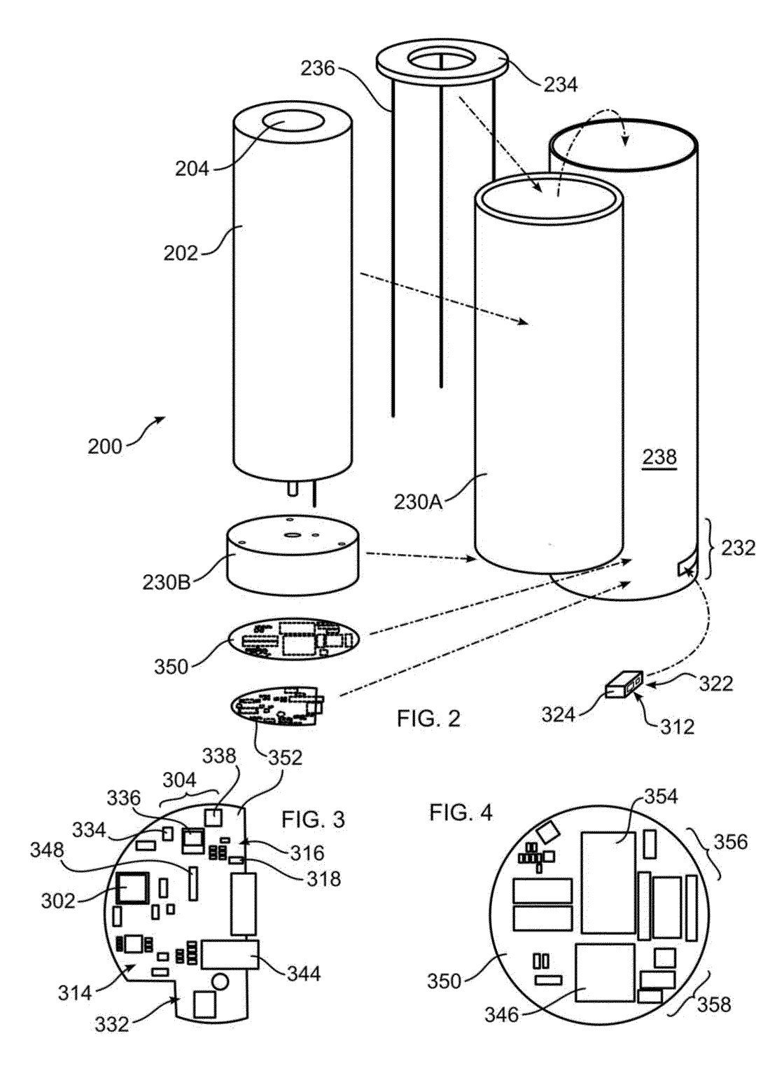

As can be seen in FIG. 2, chamber 200 includes a shielding sleeve 230A, which in an embodiment is fabricated with lead and is one-eighth inch thick, the sleeve configured to surround and shield the sides of atmospheric chamber 202 and well 204, and additionally electronics 300. Shielding sleeve 230A can be formed as a tube by extrusion, for example. A shielding plate 230B, fabricated, for example of lead and having a thickness of one inch, is inserted within the sleeve to be disposed between the atmospheric chamber 202 and electronics 300. The thicknesses of shielding in the embodiment shown are illustrative, and are selected based upon the effectiveness of the shielding material, and the anticipated maximum radiation exposed to system 100.

As may further be seen in FIG. 2, atmospheric chamber 202 is inserted within shielding sleeve 230A, and shielding plate 230B is positioned below chamber 202, thereby shielding circuits 350, 352 from radiation.

It is further a requirement to prevent electromagnetic interference (EMI) or radio frequency interference (RFI) by the emission of such energy from electronic devices, such as electronics 300. To reduce these emissions, the interior of, at least, space 232 can be coated with a conductive material which routes this energy to ground, such as a painted coating containing graphite, nickel, copper, or silver. The coating can likewise reduce the possibility of EMI or RFI from external sources adversely affecting the operation of electronics 300.

Chamber 200 can be secured within shielding sleeve by any known means. In the embodiment shown, a top plate 234 seats against an upper surface of sleeve 230A, and has a plurality of rods extending downwards. Shielding plate 230B is press fit, adhered, or otherwise secured into a position within shielding sleeve, and includes threaded apertures which are connectable to rods 236 to form a clamp for securing chamber 200. A protective and supporting outer sleeve 238 can be placed over shielding sleeve 230A, creating a covered area 232 which is below plates 230B, and within which one or more circuit boards 350, 352 containing electronics 300 are located. A protecting and sealing plate can be secured over the bottom of sleeve 230A, concealing and further protecting electronics 300, and can include a gasketed seal to provide a humidity and moisture barrier. Either the sleeve 238 and/or the bottom plate can be fabricated with, for example, plastic or metal, such as aluminum. A replaceable connector panel 324 can be attached to an exterior of sleeve 238. Wires extend from circuit boards 350, 352, though an opening in sleeve 230A adjacent circuit boards 350, 352, to Ethernet connector 312 and USB connector 322.

With reference to FIGS. 3 and 4, two different circuit boards 350, 352 are illustrated. It should be understood that a plurality of circuit boards can be advantageous for packaging, cost, and other considerations; however, electronics 300 can be implemented on a single circuit board, and thus the various circuits described herein can be present on a single circuit board, or more than two circuit boards. Further, the distribution of electronic circuits or subunits can be different than the embodiment shown in the example of FIGS. 2 and 3.

With reference to FIG. 3, in an embodiment of the disclosure, a CPU circuit board 352 can include processor circuitry 302, data storage circuitry 304, Ethernet interface circuitry 314, PoE circuitry 332, USB interface circuitry 316. Data storage circuitry 304 can include serial FLASH memory 334, an SD card or other removable storage 336, and EEPROM storage 338. A USB circuit connector 318 is connectable to USB connector 322. An Ethernet circuit connector 344 is connectable to Ethernet connector 312. A battery 348 can be provided to maintain functioning of all or portions of data storage circuitry 304 when power is not provided to board 352.

With reference to FIG. 4, an iometer circuit board 350 includes input amplifier circuitry 354, serial interface circuitry 356, power supply circuitry 358, and high voltage power supply circuitry 346. Components of board 350 are primarily directed to the operation of chamber 200, with serial interface circuitry 356 directed to communication with board 352. More particularly, serial interface circuitry 356 is connected to chamber connector 358 on board 352, and enables processor 302 to control functions of board 350, and to obtain data derived from chamber 200 operations. Serial interface circuitry can support any serial interface of sufficient bandwidth, and in an embodiment, provides an RS485 serial interface. An analog to digital circuit is provided on either the iometer board or the CPU board to convert the electrical signal representative of the extent of nuclear radiation in well 204 to a corresponding digital signal suitable for processing by processor 302. It should be understood that processor 302 may be a single processor, or a plurality of individual processor components or CPUs.

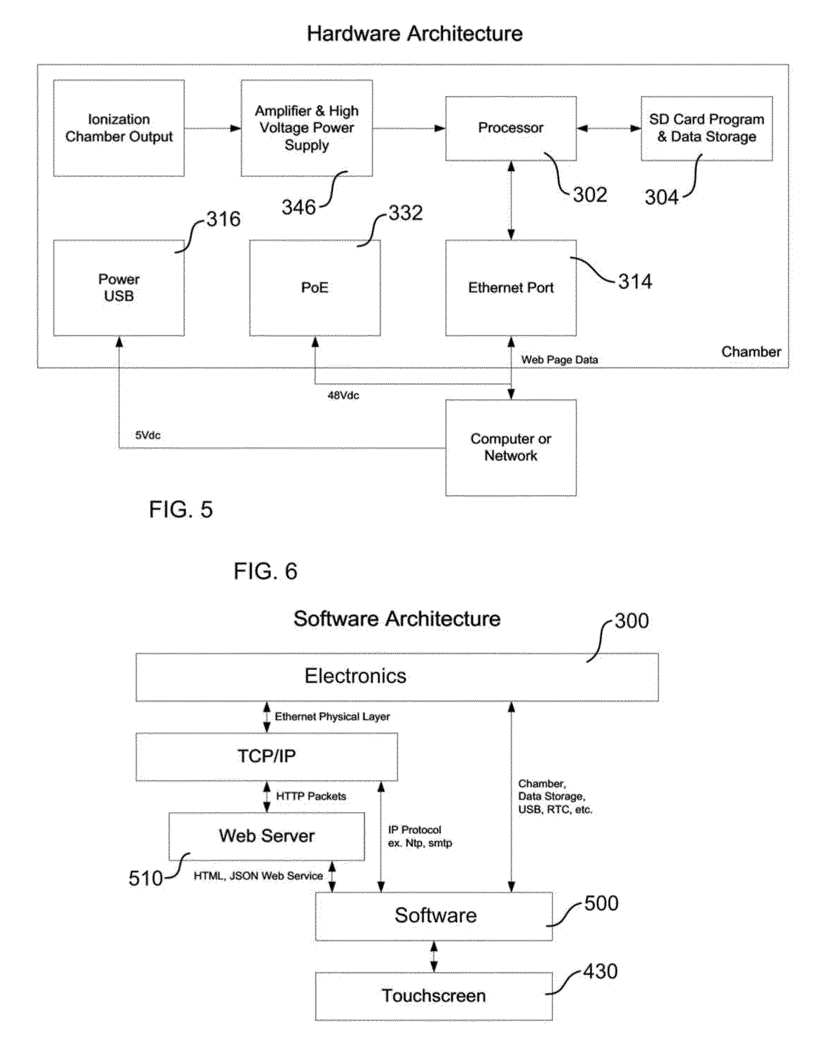

FIG. 5 diagrammatically represents the hardware architecture of system 100 physically located within the chamber structure illustrated in FIG. 2. It should be understood, however, that certain electronic functions carried out within this structure can be externalized, for example in an external housing connected by a cable or a wireless connection, such as BLUETOOTH. With further attention to FIG. 5, and more particularly, ionization chamber 200 output is directed to an amplifier and high voltage power supply 346, the output of which is converted to a digital data and this chamber data is sent to processor 302. Stored values, for example from SD card 336 or other memory or storage 304, are used by processor 302 to process the chamber data, as described elsewhere herein. Processor 302 communicates to a computer or network using either Ethernet port 312, or USB port 322. Power is obtained for all operations within chamber 200 by PoE and/or USB.

In FIG. 6, a software architecture for system 100 is diagrammatically depicted. Electronics provides an interface to LAN and WAN networks, provides data storage, communication and power interfaces 310, 320, processing, a real time clock (RTC), and other resources relied upon by software 500 executing upon processor 302, which can include a plurality of processors and/or microcontrollers. Software 500 of the disclosure may programmatically control a dedicated touchscreen 430 connected to system 100 by a wired or wireless connection. Touchscreen 430 can correspond to a display with a touch sensitive surface enabling the input of data, for example using a displayed keyboard. Touchscreen 430 can alternatively include a physical keyboard, touchpad, or other input device, and can have any or all of the capabilities of a laptop computer or other general purpose computing device.

Software 500 further has access to the Ethernet network, which provides TCP/IP communication to a LAN or WAN. As such, software 500 can exploit various protocols available via TCP/IP, such as the Network Time Protocol (Ntp) for calibrating real time, Simple Mail Transfer Protocol (SMTP) for communication using email, or any other protocol useful for carrying out the functionality described herein. A component of software 500 is web server 510, which carries out communication with a computing device on the LAN or WAN to which system 100 is connected. Typically, web server 510 communicates using HTTP packets, although other protocols can be supported, including FTP, for example. Web server 510 can access data from processor 302, and from storage 304, using HTML, and server side technologies such as JSON, PHP, or Ruby, for example.

Software 500 can further access other data that can be provided by electronics 300, including data derived from operation of chamber 200. Further, software 500 can control the operation of chamber 200, including regulating the electric field, and analyzing electrical effects of radiating materials placed into chamber 200.

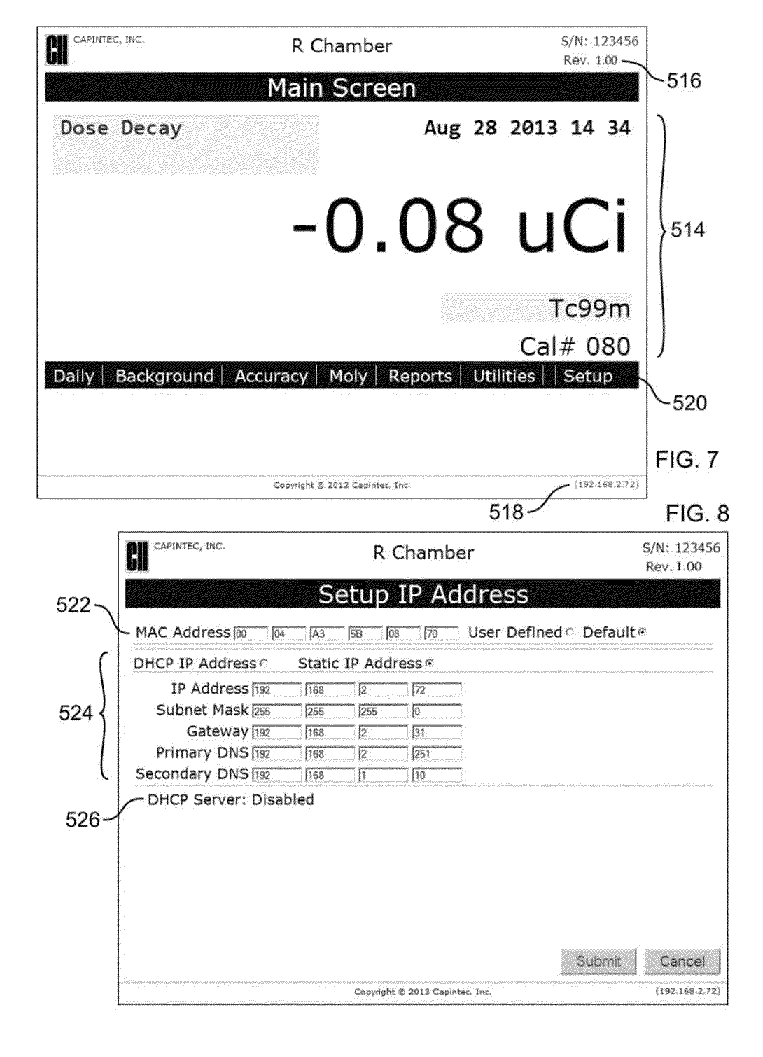

Referring now to FIGS. 7-9, using a touchscreen 430, or a connected computing device 400, a user may execute a browser application and direct the browser to the IP address of system 100. If initially setting up system 100, computer 400 can be directly connected to system 100, and can be configured to communicate on the same subnet as a default subnet of system 100. A default or preestablished IP address of system 100 is provided, which can be entered as a URL in the browser, so that the browser can access the web server 510 features of board 352.

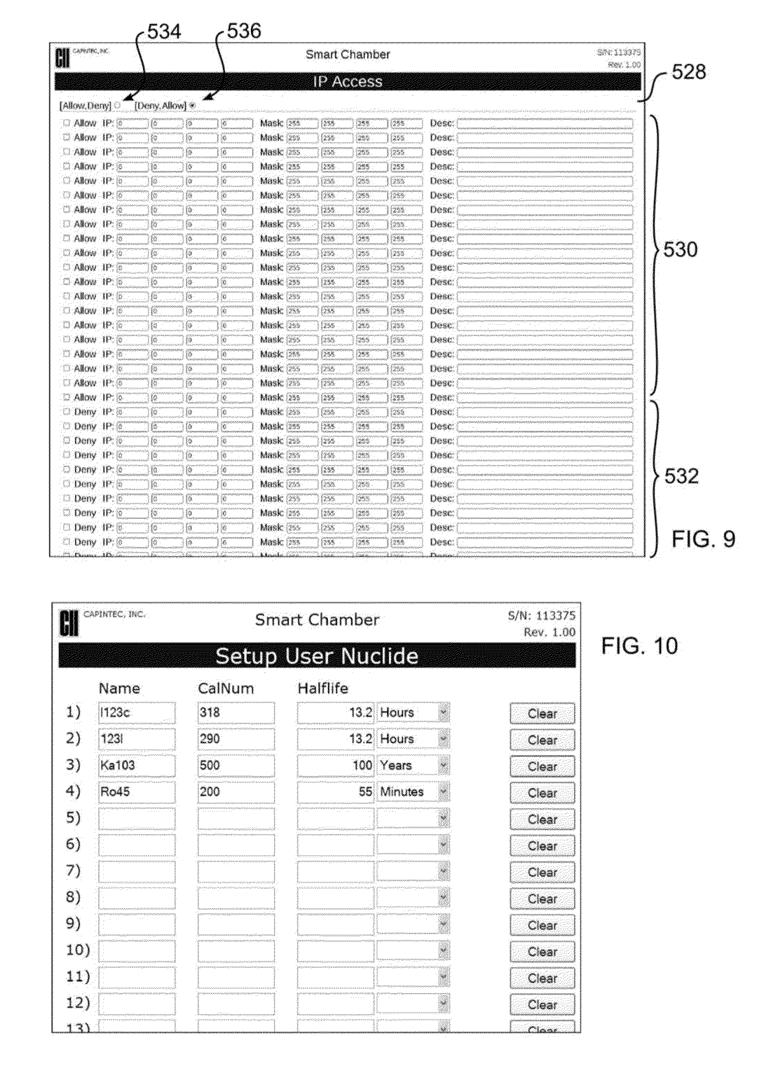

Once connected, an initial screen is displayed as can be seen in FIG. 9. Parameters 514 are displayed indicating a currently selected test, as well as the date, time, and information pertaining to Dose Decay and current radiation intensity. A revision number 516 of software 500 is indicated, as well as the current IP address (518) of system 100. In an embodiment, the current IP can be used by other computers on the LAN to access a particular system 100. When connecting by a WAN connection, it may be necessary in certain configurations to establish a port redirection or other route for this connection in a WAN router. A menu 520 of options is additionally displayed, including, in this embodiment, the options Daily, Background, Accuracy, Moly, Reports, Utilities, and Setup.

Selecting the Setup menu 520 option causes software 500 to render the display shown in FIG. 8, in which it is possible to set the MAC Address 522 of system 100, or choose the default MAC address. Changing the MAC address can simplify MAC address filtering in switches, routers, or other electronic controls on a network, where only devices having MAC addresses matching specific criteria are allowed to communicate using the network. Additionally, setting the MAC address can simplify recordkeeping, identification, organization, and MAC based authentication in an installation with numerous instances of system 100. The MAC address can further be changed periodically to improve security. It may further be seen that the IP parameters 524 may be set automatically using DHCP, or can be set manually to conform to a network environment in which system 100 will be used. In this manner, system 100 can be made visible on the network by a plurality of computing devices 400 which are on the same subnet, for example, or may alternatively be placed on a subnet with access that is limited for safety and security.

System 100 can additionally serve as a DHCP server 526, providing IP addresses to one or more connected computing devices. In an embodiment, when DHCP server is enabled, for increased security, system 100 provides only a single address to a single connected computing device 400, and only provides this address during the first three minutes that system 100 is powered up. Further, this mode can only be enabled from a computer connected by a USB cable. In this manner, the possibility of tampering or misuse is reduced.

More particularly, in one embodiment, electronics 300 and software 500 are configured to enable access to this DHCP server mode in accordance with the following steps:

1. Enable the DHCP server mode:

a. Connect system 100 to a computer using USB interface 320.

b. Using the MICROSOFT WINDOWS Device Manager application, determine the COM port of system 100.

c. Send the Enable command to system 100 as follows:
i. using the Windows DHCP Server Utility, select the COM port from the COM Port drop-down box. Click the Enable DHCP Server button. (To disable the DHCP Server, click the Disable DHCP Server button.)

OR
ii. use a serial terminal program, such as PUTTY or Hyper Terminal. Connect the terminal program to the COM port of system 100, and type “dhcps on” into the terminal program. System 100 will reply with “DHCP Server: Enabled”. (To Disable DHCP Server, type “dhcps off” into the terminal program.)

2. Remove power from system 100.

3. Connect system 100 Ethernet port 312 directly to a DHCP client (Windows™ PC).

4. Power up system 100.

5. Wait for the DHCP client to recognize system 100.

6. The IP address of system 100 will be one less than the address of the computer. In Windows, the PC's IP address can be obtained by typing “ipconfig” into a command window.

Additionally, this mode can be used as a fail-safe mode. If the IP Settings/Access were incorrectly configured, then this mode can be used to access system 100 to reconfigure the IP Settings/Access.

In FIG. 9, it may be seen that system 100 provides access control by IP address 528, using the browser interface 502 of system 100. In the embodiment shown, there are a plurality of possible ‘Allow’ IP address ranges 530, and a plurality of possible ‘Deny’ IP address ranges 532. In this embodiment, an IP range is activated by selecting the ‘[Allow, Deny]’ radio button and entering the desired IP address and subnet Mask, and optionally entering a description.

In one embodiment, entering a 0 as the last octet in the address designates the entire possible range from 0.0 to 0.255. In another embodiment, an asterisk can be used in the IP address to indicate a range. As an example: IP: 192.168.2.0 and Mask: 255.255.255.0 defines IP Ranges 192.168.2.0-192.168.2.255. Alternatively, a single IP address can be designated by using a non-zero value in the last octet, for example IP: 192.168.2.13 and Mask 255.255.255.255 defines one IP Address 192.168.2.13.

System 100 can additionally implement a predefined set of devices with which it will communicate over an IP network. The ‘Allow, Deny’ radio button 534 enables system 100 to evaluate the ‘Allow’ IP ranges first, and then the ‘Deny’ IP ranges. In this instance, if an address is not in the ‘Allow’ list, it will be denied. This is a useful setting if there are only a limited number of addresses for which access is desired. For this reason, the ‘Deny’ list is used to deny a subset of addresses that are in the ‘Allow’ list, for example which are allowed as part of a range.

The ‘Deny, Allow’ radio button 536 enables system 100 to evaluate the ‘Deny’ IP ranges first and then the ‘Allow’ IP ranges. This is a useful setting of there are only a limited number of addresses for which access is not desired. In this instance, if an address is not in the ‘Deny’ list, it will be allowed. For this reason, the ‘Allow’ list is used to allow a subset of addresses that are in the ‘Deny’ list, for example which are denied as part of a range.

With reference to FIG. 10, in addition to storing a MAC address, Static IP information, and DHCP server info, including the set of allowed devices, data storage 304 can further store information pertaining to quality control testing results, Moly tests, and User Logs. In addition, data storage 304 can store information regarding a plurality of frequently used nuclides, for example 80, as well as a plurality of user defined nuclides, for example 30, limited only by available memory.

In FIG. 10, a user can enter a Name, CalNum, and Halflife, in for each user Nuclide, in accordance with the following.

The Name Field should correspond to the nuclide designation (i.e. Tc99m, Cs137) and can consists of up to 6 alphanumeric characters.

The CalNum is the Calibration Number for the nuclide (i.e. 080). It may include a multiplication sign (* on the keyboard) or a division sign (/ on the keyboard). However, in an embodiment, system 100 is always direct reading and the multiplication or division sign is only used to be consistent with existing Calibration Numbers. In one embodiment, for multiplication, the number can only be multiplied by 10 or 100. For division, the number can only be divided by 2. It should be understood that these values are provided for the illustrated embodiment, and that in other embodiments, other values can be used. In one embodiment, calibration limits are as shown in Table 1. In another embodiment, a calibration number range is 10 to 990, adjusted by multipliers or dividers when the calibration number is outside the range.

TABLE 1
Example Calibration Number Limits

| | Minimum | Maximum |
|---|---|---|
| | Calibration # (a) | Calibration # (a) |
| Direct Entry (a) | 10 | 1200 |
| Multiplication (a × 10) | 10 | 1200 |
| Multiplication (a × 100) | 10 | 999 |
| Division (a ÷ 2) | 400 | 1200 |

An initial determination of Calibration Number is input into system 100 for the nuclide. As an initial starting point, one can choose 450. Note: In order to obtain a correct reading for a Vial or Syringe, the supplied Liner and Dipper must be used to achieve the correct geometry. If the source is contained in a different type of container, the manufacturer of system 100 can be contacted for obtaining the correct values for the container.

1. Place the standard source of the nuclide in system 100 and record the displayed activity.

2. If the displayed activity is higher than the measured/calculated activity of the standard source, increase the Calibration Number. If the displayed activity is lower than the measured/calculated activity of the standard source, decrease the Calibration Number.

3. Re-measure the activity of the standard source.

4. Continue to increase or decrease the Calibration Number (e.g. repeat steps 2 and 3) until the displayed activity matches the measured/calculated activity of the standard source.

5. Record the Calibration Number of the nuclide for future reference.

6. Input the new Calibration Number.

The Halflife is the half-life of the nuclide (i.e. 6.01 hr, 30.00 yr) and consists of up to 6 characters (5 digits and a decimal). Input the value using the keyboard and then click on the drop-down list box. From the drop-down list, click the desired time unit for the halflife. The available time units for halflife are: Minutes, Hours, Days, and Years

Processor 302 and software 500 are additionally configured to calculate decay for references sources stored in memory, based on a current date. An accurate date can be obtained automatically from the internet. Additionally, automated dose calibration, quality control tests, and self-diagnostics are carried out by processor 302 and software 500, and background radiation can automatically be subtracted from measurements, facilitating use of system 100. Additionally, data is formatted for output to a printer or other output device, and can be printed, for example, to conform to NRC records requirements. Further, processor 302 and software 500 are further configured to communicate with Nuclear Medicine Management systems by Ethernet or USB interfaces 310, 320, respectively. Software 500 can further be upgraded using the USB or Ethernet interface 320, 310, and the process can be carried out using the internet in an automated manner, without requirement of user intervention.

In an example embodiment, chamber 200 has these approximate physical parameters: height 45.8 cm, diameter 17.2 cm, weight 17.8 kg, well diameter 6.1 cm, well depth 25.4 cm, PoE cable length 3.7 m. It should be understood that system 100 can be used with a chamber 200 of any dimension or type. In an embodiment, electronics 300 include an electrometer having an accuracy better than ±2%, linearity within ±2%, and a response time within 2 sec. (4 to 16 sec. for very low activity samples).

Repeatability is within ±1% within 24 hours, during which time the calibrator is on all the time. System 100 is configured for a full test of program 500 and system memories 304, and for daily test for auto zero, auto background adjust, data check, accuracy and constancy, and voltage test. Standard source data can be provided, and in an embodiment, source data for standard sources of Co-57, Co-60, Ba-133, Cs-137, Na22 are preconfigured.

System 100 is configured for Molybdenum-99 Assay testing, using the Canisters or CAPMAC method, measuring values for Mo-99 elution, Tc-99m, and Tc-99m/Mo-99 Ratio.

Example Computing System and Components

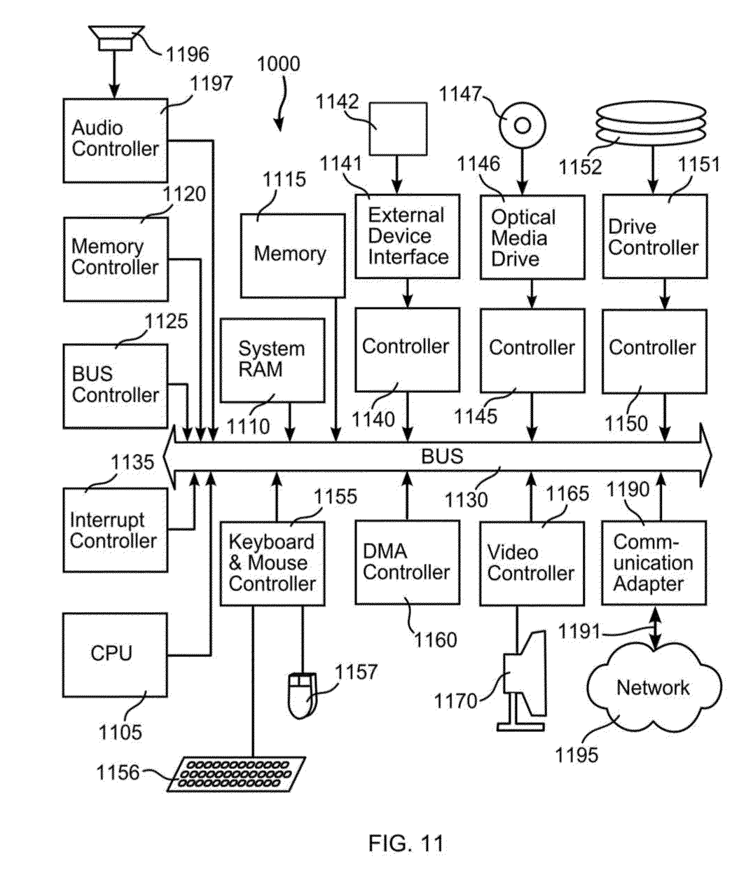

FIG. 11 illustrates a system architecture for a computer system 1000 that can be used along with system 100, or which contains one or more components which can be included within electronics 300, in various embodiments thereof. The example computer system of FIG. 11 is for descriptive purposes only. Although the description may refer to terms commonly used in describing particular computer systems, the description and concepts equally apply to other systems, including systems having architectures dissimilar to FIG. 11. Computer system 1000 can control a wide variety of electrical devices, including electrodes, sensors, and detectors. One or more sensors, not shown, provide input to computer system 1000, which executes software stored on non-volatile memory, the software configured to received inputs from sensors, electrodes, or from human interface devices, in performing calculations for controlling system 100.

Computer system 1000 includes at least one central processing unit (CPU) 302, or server, which may be implemented with a conventional microprocessor, data storage 304 which can include random access memory (RAM) 1110 for temporary storage of information, and a read only memory (ROM) 1115 for permanent storage of information, as well as FLASH memory 334, an SD card or other removable storage 336, and EEPROM storage 338. A memory controller 1120 is provided for controlling RAM 1110 or any other data storage device.

A bus 1130 interconnects the components of computer system 1000. A bus controller 1125 is provided for controlling bus 1130. An interrupt controller 1135 is used for receiving and processing various interrupt signals from the system components.

Mass storage may be provided by DVD ROM 1147, or flash or rotating hard disk drive 1152, for example, or any other data storage device, whether mechanical or solid state. Data and software, including software 500 of the disclosure, may be exchanged with computer system 1000 via removable media such as diskette, CD ROM, DVD, Blu Ray, or other optical media 1147 connectable to an Optical Media Drive 1146 and Controller 1145. Alternatively, other media, including for example a media stick, for example a solid state USB drive, or SD card 336, may be connected to an External Device Interface 1141, and Controller 1140. Additionally, a system 100 in accordance with the disclosure may be connected to computer system 1000 through Ethernet or USB Interfaces 310, 320, respectively, or with a BLUETOOTH, Infrared, or WiFi connector, although other modes of connection are known or may be hereinafter developed. A hard disk 1152 is part of a fixed disk drive 1151 which is connected to bus 1130 by controller 1150. It should be understood that other storage, peripheral, and computer processing means may be developed in the future, which may advantageously be used with the disclosure.

User input to computer system 1000 may be provided by any of a number of devices. For example, a keyboard 1156 and mouse 1157 are connected to bus 1130 by controller 1155. An audio transducer 1196, which may act as both a microphone and a speaker, is connected to bus 1130 by audio controller 1197, as illustrated. It will be obvious to those reasonably skilled in the art that other input devices, such as a pen and/or tablet, Personal Digital Assistant (PDA), mobile/cellular phone and other devices, may be connected to bus 1130 and an appropriate controller and software, as required. DMA controller 1160 is provided for performing direct memory access to RAM 1110. A visual display is generated by video controller 1165 which controls video display 1170. Computer system 1000 also includes Ethernet and USB communications interfaces 310, 320 which allow the system to be interconnected to a local area network (LAN) or a wide area network (WAN), schematically illustrated by bus 1191 and network 1195 in FIG. 11, and/or as illustrated in FIG. 4.

Operation of computer system 1000 is generally controlled and coordinated by operating system software, such as a Windows system, commercially available from Microsoft Corp., Redmond, Wash., or an embedded system, for example based on the LINUX operating system. The operating system controls allocation of system resources and performs tasks such as processing scheduling, memory management, networking, and I/O services, among other things. In particular, an operating system resident in system memory and running on CPU 1105 coordinates the operation of the other elements of computer system 1000. The present disclosure may be implemented with any number of commercially available operating systems.

One or more applications, such as an HTML Web page server 510, or a commercially available communication application, may execute under the control of the operating system, operable to convey information to a user.

All references cited herein are expressly incorporated by reference in their entirety. It will be appreciated by persons skilled in the art that the present disclosure is not limited to what has been particularly shown and described herein above. In addition, unless mention was made above to the contrary, it should be noted that all of the accompanying drawings are not to scale. There are many different features to the present disclosure and it is contemplated that these features may be used together or separately. Thus, the disclosure should not be limited to any particular combination of features or to a particular application of the disclosure. Further, it should be understood that variations and modifications within the spirit and scope of the disclosure might occur to those skilled in the art to which the disclosure pertains. Accordingly, all expedient modifications readily attainable by one versed in the art from the disclosure set forth herein that are within the scope and spirit of the present disclosure are to be included as further embodiments of the present disclosure.

What is claimed is:

1. A method for measuring nuclear radiation in a material, comprising:
converting radiation from the material that has been inserted into a chamber into an electrical signal corresponding to an extent of nuclear radiation in the material;
using a first electronic circuit connected to the chamber for powering the chamber with an electrical signal, and for receiving an electrical signal from the chamber in response to the presence of radiation in the material;
processing the received electronic signal using an electronic processor executing software stored on non-transitory media, the software operative to process the digital data;
storing the processed digital data using a data storage device connected to the processor;
connecting the processor to a electronic communication network; and
hosting a web server, using the processor and software, the web server operative to generate and host pages for access through the network, the pages corresponding to the processed digital data.

2. The method of claim 1, wherein the processor is connected to an electronic circuit operative to communicate with an Ethernet network.

3. The method of claim 1, wherein the network is an Ethernet network.

4. The method of claim 1, wherein the network is a TCP/IP network.

5. The method of claim 1, wherein the network is a USB Network.

6. The method of claim 1, further including providing radiation shielding positioned between the chamber on a first side, and the first electronic circuit and processor on a second side opposite the first side.

7. The method of claim 1, wherein the network is an Ethernet network, and further including a circuit for obtaining electrical energy sufficient for the operation of the chamber, the first electronic circuit, and the processor, when the Ethernet circuit is connected to a circuit providing Power Over Ethernet (PoE).

8. The method of claim 1, wherein the material is a therapeutic dosage.

9. A system for measuring nuclear radiation in a material, comprising:
a chamber into which the material may be inserted, the chamber configured to convert an extent of nuclear radiation in the material into a corresponding electrical signal;
a first electronic circuit connected to the chamber and including a circuit for powering the chamber with an electrical signal, and a circuit for receiving an electrical signal from the chamber in response to the presence of radiation in the material and for generating an output signal;
a second electronic circuit connected to the first electronic circuit and including:
an electronic processor operative to process the output signal into processed data;
data storage connected to the processor and operative to store processed digital data;
an electronic communication network connected to the electronic processor; and
software stored on non-transitory media, the software executable by the processor and configured for:
software executable upon the electronic processor and configured to provide a web server operative to generate and host pages for access through the network, the pages corresponding to the processed digital data.

10. The system of claim 9, wherein the software is further configured for generating one or more pages with the web server enabling a configuration of the system to allow or deny access to hosted pages of the system based upon a network address of one or more computers attempting to access the hosted pages using the network.

11. The system of claim 9, wherein the circuit for receiving the electrical signal from the chamber further includes circuitry for converting the received electrical signal to digital data, the digital data corresponding to the output signal.

12. The system of claim 9, wherein the network is a Power Over Ethernet (PoE) network, and where the first and second circuit derive all required operating power through power provided by the PoE network.

13. The system of claim 9, further including providing radiation shielding positioned between the chamber on a first side, and the first and second electronic circuits on a second side opposite to the first side.

14. The system of claim 9, further including a circuit for obtaining electrical energy sufficient for the operation of the system when the network is a circuit providing Power Over Ethernet (PoE).

15. The system of claim 9, further including a USB circuit operative to enable the processor to communicate by USB.

16. The system of claim 9, further including a USB circuit operative to obtain electrical energy sufficient for the operation of the system when the USB circuit is connected to a source of power.

17. The system of claim 9, wherein the first electronic circuit includes iometer circuitry.

18. The system of claim 9, wherein the circuit for receiving the electrical signal includes input amplifier circuitry.

19. The system of claim 9, wherein the circuit for powering the chamber includes high voltage power supply circuitry.

20. The system of claim 9, wherein the first circuit and the second circuit are connected by a serial interface.
